(19) United States
(12) Patent Application Publication
(10) Pub. No.: US 2008/0167626 A1
(43) Pub. Date: Jul. 10, 2008

(54) CATHETER COVERING FOR USE IN BATHING

(76) Inventors: Gunning T. Beery, Longs, SC (US); Christopher Po, Longs, SC (US)

Correspondence Address: LAW OFFICE OF BRETT N. DORNY, 386 WEST MAIN STREET, SUITE 12A, NORTHBOROUGH, MA, 01532, US

(21) Appl. No.: 11/623,912

(22) Filed: Jan. 17, 2007

Related U.S. Application Data
(60) Provisional application No. 60/759,515, filed on Jan. 17, 2006.

(51) Int. Cl. A61M 25/00

(52) U.S. Cl. 604/263

(57) ABSTRACT

A catheter covering is disclosed for use during a shower or bath. The covering includes a receptacle formed of two layers of a membrane. A hole is formed in one layer. An adhesive strip is positioned around the hole. The adhesive strip is of a type which provides a water-tight seal for a short duration and then is easily detached. The color of the receptacle may be selected to represent the function of the catheter being protected.

### CROSS-REFERENCE TO RELATED APPLICTIONS

This application claims priority to U.S. Provisional Patent Application No. 60/759,515, filed Jan. 17, 2006.

### BACKGROUND OF THE INVENTION

1. Field of the Invention

The present invention relates to a protective device for a catheter or other medical device which extends through the skin into the body. More particularly, it relates to a simple protective device to keep moisture and matter from the input site of the catheter.

2. Discussion of Related Art

Intravenous catheters are typically inserted into patient's arms or body in order to administer medications or for use in dialysis for attaching dialysis patients to blood purification units in order that contaminates can be removed from the patient's blood. These catheters remain inserted into the patient's arms for extended durations. It is critical to keep the catheter free from contaminants when not in use. These contaminants allow bacteria to grow and propagate and become a risk of infection to the patient. Additionally, subsequent utilization of the catheter for dialysis or administration of medication via the catheter could result in blood contamination. Many potential contaminants come from water. Thus, the catheter must be kept dry in order to minimize contamination.

In the past, catheters have been taped to the arm with a plastic bag and cellophane tape in an attempt to keep out water and contaminants with limited success. In addition, the removal of the cellophane tape can sometimes damage the catheter or seriously irritate the patient's skin. A better method is needed for keeping intravenous catheters and associated areas dry and free of contaminants and a method that is easy to attach and easy and less painful to remove from the patient's skin. The current state of the art of attached devices for keeping surgical areas dry are made to cover incisions for surgery and severely adhere to the skin of the patient and are not easily removed.

Typically, persons with intravenous catheters implanted have difficulty taking showers or baths. Many of the potential contaminants come from water and shower or bath products. Consequently, sponge baths are common in this situation. These sponge baths are inconvenient for the patient and time consuming for compared to taking a quick shower. The Applicants' device would alleviate the problem of typically having to keep the area with the intravenous catheter dry while bathing. After the shower or bath, applicant's device can be easily removed and the medical staff may administer medication or start blood dialysis through a non-contaminated catheter.

A number of devices have been created and patented to prevent catheter contamination with various levels of success. U.S. Pat. No. 6,222,090 discloses a waterproof covering for a injection area. An adhesive is provided around the outer edges of the waterproof covering to attach it to the patient. The waterproof covering may have a raised area to keep it away from the covered area of the body. In order to accommodate a catheter and tubing, the waterproof covering of this design either has to be significantly large or significantly deep.

U.S. Pat. No. 6,124,521 discloses a dermal wound dressing for use with a catheter. It is designed to allow the tubing to extend through the dressing. It requires multiple adhesive parts to provide a seal, which can cause additional skin irritation. It further requires an absorbent layer as part of the seal.

U.S. Pat. No. 5,605,546 discloses a receptacle for protecting a catheter or similar device. The receptacle includes an adhesive around an inlet through which the ends of the catheter are inserted into the receptacle. An outlet, with a separate covering, is used to access the ends of the catheter through the receptacle. The receptacle is maintained in place for long periods of time between individual or multiple treatments. Thus, it is a long term, strong binding type which can significantly irritate the skin.

Most devices for protecting the catheter have complicated structures necessary for sealing the area and are difficult to utilize. They cannot be applied quickly in order to take a shower or bath and may require two people to install. Furthermore, they are difficult to remove. Most such devices are intended for long term application, for a day or multiple days. Such adhesives irritate the patient's skin.

### SUMMARY OF THE INVENTION

The present invention provides a simple device which is easily attached to the patient around the catheter to protect it from contamination during bathing. The device includes a multiple layer, non-permeable polymeric membrane which functions in a tent like fashion. An opening in one of the layers allows the external ends of the catheter to be inserted within the membrane. An adhesive strip is affixed to the edges of the opening. The adhesive is of a type that adheres completely to the patient's skin making a water tight seal against contaminants and specifically water. The adhesive is also of a kind that attaches securely for short duration and then allows the device to be detached from the patient's skin with minimal reaction to disruption to the skin surface.

### BRIEF DESCRIPTION OF THE DRAWINGS

FIG. 1 is a front view of a catheter covering device according to an embodiment of the present invention.

FIG. 2 is a cross sectional view of the catheter covering device of FIG. 1.

### DETAILED DESCRIPTION

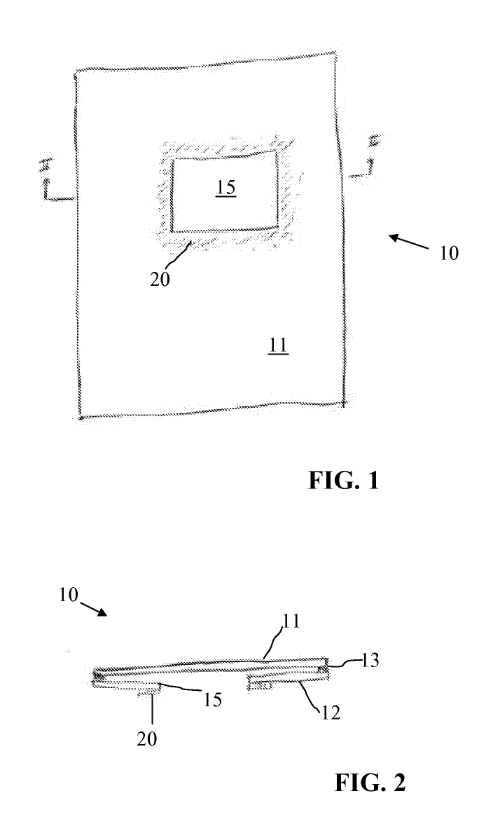

A device according to the present invention is used to cover an intravenous catheter. The device 10, illustrated in FIGS. 1 and 2, is removable and temporarily attached to the skin of the patient fully covering and enclosing an intravenous catheter in a non-permeable membrane that will keep out water and other contaminants during a shower or bath.

As illustrated in FIGS. 1 and 2, the device 10, includes a receptacle formed by two layers 11, 12 of a flexible and pliable, non-sterile, non-permeable membrane. The membrane may be made of one of a number of materials safe for use in medical situations for public consumption. Examples are clear or opaque plastics and/or polymers, etc. In choosing the material for this application, one would take into consideration the specific application or intended use; age of the patient; and a patient's possible allergies. The two layers 11, 12 of the membrane are sealed 13 along the outside edges. Depending upon the material used, the membrane layers 11, 12 may be heat sealed, sealed with an adhesive or in some other manner. The seals 13 on the edges of the membrane layers 11, 12 should be permanent and water impervious.

One membrane layer 12 has a hole 15 formed in a portion thereof. The hole 15 is sized to accommodate the external portion of the catheter. The device 10 and hole 15 may be sized based upon the type of catheter used. Alternatively, they may be sized to accommodate different types of catheters.

An adhesive strip 20 surrounds the hole 15 in the one membrane layer 12. The two layers 11, 12 of the membrane makes a sort of tent or receptacle body that covers the catheter. The adhesive strip 20 is used to attach the device 10 to the skin of the patient. Preferably, the adhesive strip 20 is of a type which provides a short duration, water-tight seal. After the short duration, the adhesive can be easily removed from the skin. Thus, the device 10 temporarily encases the catheter in a water-tight membrane and keeps the catheter free of contaminants while the patient can take a shower or bath. The device 10 can be easily removed with minimal irritation to the skin of the patient. The adhesive may be varied in order to accommodate different types of environmental situations and/or applications and different types of skin, or modified to accommodate skin allergies in patients.

The dimensions of the device can be varied in order to accommodate catheters of different sizes and/or patients of different ages. According to an embodiment of the invention, the device 10 is approximately 5 inches by 7 inches. The hole 15 is approximately 2 inches by 3/2 inches. The adhesive strip 20 is approximately 4 inches by 4 ¾ inches. According to another embodiment of the invention, the device 10 is approximately 6 inches by 8 inches; the hole is 3/2 inches square; and the adhesive strip is approximately 4/2 inches square. According to another embodiment, the hole is round with a 3 inch diameter. Of course, any other dimensions could be used. Furthermore, the device 10 and the hole 15 have been illustrated as rectangular in shape. Any other shape, including circles or ovals, could be also be used for the device 10 or the hole 15.

Also, the color of the receptacle body or the adhesive strip could be varied to indicate the function of the catheter. For example, dialysis patients could have a specific colored non-permeable membrane, receptacle body.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A device for covering an intravenous catheter or feeding tube inserted into a patient to keep out water and contaminants during bathing, the device comprising:
a two layer receptacle formed of a non-permeable membrane, one layer of the receptacle having a hole therein; and
an temporary adhesive on one side of the receptacle positioned around the edges of the hole in one layer of the receptacle.

2. The device for covering an intravenous catheter according to claim 1, wherein the membrane is of a flexible, non-porous, clear or opaque, non-sterile plastic.

3. The device for covering an intravenous catheter according to claim 1, wherein the membrane is of a flexible, non-porous, clear or opaque, non-sterile polymer.

4. The device for covering an intravenous catheter according to claim 1, wherein the adhesive is of a type providing a water-tight seal of a short duration.

5. The device for covering an intravenous catheter according to claim 1, wherein the device is shaped based upon the catheter.

6. The device for covering an intravenous catheter according to claim 1, wherein the membrane is colored.

7. The device for covering an intravenous catheter according to claim 6, wherein a color is selected based upon the function of the intravenous catheter.

8. The device for covering an intravenous catheter according to claim 1, wherein the adhesive is non-allergenic.
